(19) United States
(12) Patent Application Publication
(10) Pub. No.: US 2016/0317790 A1
(43) Pub. Date: Nov. 3, 2016

(54) Balloon Catheter

(71) Applicant: Alexander Ruebben, Monaco (MC)

(72) Inventor: Alexander Ruebben, Monaco (MC)

(21) Appl. No.: 15/109,071

(22) PCT Filed: Dec. 30, 2014
(86) PCT No.: PCT/EP2014/079417

§ 371 Date: Jun. 29, 2016

(30) Foreign Application Priority Data
Dec. 30, 2013 (DE) 10 2013 021 998.1

(51) Int. Cl. A61M 25/10; A61L 29/04; A61L 29/02; A61M 25/00; A61M 25/09

(52) U.S. Cl. CPC A61M 25/1006; A61M 25/104; A61M 25/0054; A61M 25/09; A61L 29/02; A61L 29/049; A61M 2025/0059; A61M 2025/105; A61M 2025/1079

(57) ABSTRACT

The invention relates to a balloon catheter comprising an outer tube (1) to which distal end a balloon (2) connects that can be expanded by feeding in a fluid via the outer tube (1), wherein an inner tube (3) extends at least partially through the outer tube (1), said inner tube (3) passing through the balloon (2) and terminating distally of the balloon (2), with the inner tube (3) having a lumen appropriate to accommodate a guidewire (6) and the balloon catheter having a distal (a,b) and a proximal section (c), wherein the proximal section (3) of the outer tube (1) is made of metal and the distal section (a,b) consists of plastic material making the distal section (a,b) more flexible than the proximal section (c), and wherein the distal section (a,b) consists of a first distal section (a) and a second distal section (b), said second distal section (b) being located proximal to the first distal section (a), with the first distal section (a) being made so as to be more flexible than the second distal section (b). The balloon catheter proposed by the invention combines in different sections materials of different flexibility which not only results in the catheter to be navigated without difficulty also over longer distances of more than 1 m but enables it as well to readily adapt to narrow-lumened blood vessels in the distal section (a,b).

The invention relates to a balloon catheter comprising an outer tube to which distal end a balloon connects that can be expanded by feeding in a fluid via the outer tube, wherein an inner tube extends at least partially through the outer tube, said inner tube passing through the balloon and terminating distally of the balloon, with the inner tube having a lumen appropriate to accommodate a guidewire and the balloon catheter having a distal and a proximal section, wherein the proximal section of the outer tube is made of metal and the distal section consists of plastic material making the distal section more flexible than the proximal section.

In medicine so-called “minimally invasive procedures” play an ever increasing role. Percutaneous transluminal angioplasty (PTA) by means of balloon dilatation is frequently employed for the treatment of vasoconstrictions such as arteriosclerosis. For this purpose, a balloon catheter provided in its distal area with an element (balloon) inflatable by the infeed of a fluid is brought to the stenotic site (vasoconstriction) with the help of a guide catheter. The balloon catheter has an infeed lumen extending through the balloon catheter up to the proximal end and adjoining to the balloon at the distal end. By feeding in a fluid the balloon is expanded by applying high pressure of at least 4 bar as a rule, however frequently of 8 to 12 bar, so that deposits in the area of the lesion are pressed against the inner wall of the artery with a view to eliminating the stenosis in this manner and improving the flow of blood. To rule out the reoccurrence of such a vessel constriction a stent may often be implanted to keep the vessel open. After the collapsing of the balloon bringing it to a smaller cross section the balloon catheter is withdrawn and removed from the vascular system whereas a stent that may have been implanted will remain in the vessel. Access to the blood vessel system of a patient is gained with the help of an insertion device usually from the groin area via the femoral artery.

To enable a catheter for the treatment of stenoses to be navigated through the blood vessel system it must meet a number of requirements. It is to be noted in this context that the forward movement often has to cover a comparatively long distance, for example if a catheter is inserted via the groin area and has to be pushed towards the intracranial region. For that reason the catheter must have a certain stiffness. On the other hand, it may just as well be necessary for a catheter to be navigated through blood vessels of narrow lumen which in the interest of keeping the blood vessel free from injury and enable the forward movement to be performed without difficulty would call for a rather soft catheter material to be employed advantageously. To be able to satisfy these basically contradictory requirements balloon catheters have already been in use that feature metal, in particular stainless steel, to be used for the proximal part whereas a distal section consists of a plastic material, for example nylon. The portion of the catheter made of stainless steel is frequently termed hypotube. In this way, a catheter is obtained that on the one hand has relative stiffness especially in its proximal section so that pushing distances of more than 1 m can also be covered. What is more, due to the stiffness of the hypotube torque forces may be transmitted as well. On the other hand, the distal section of the catheter, which is typically significantly shorter than the metallic proximal section, is made of a comparatively soft plastic material which enables the catheter to follow even narrow convolutions of blood vessels without problems especially in the distal area. Moreover, using for the distal section a polymer material also offers protection for the catheter to prevent undesirable kinking or buckling. Such kinking or buckling that may occur as a result of the poor adaptability of a purely metallic balloon catheter may also cause the forward movement of the catheter to be impeded and practically rendered impossible so that the catheter will become useless. Moreover, there is the imminent risk of the inner wall of the blood vessel to be injured due to kinking/buckling, in particular if the catheter coils up at the kinking location as the pushing force continues to be exerted.

Due to the measures described hereinbefore the balloon catheters manufactured by prior art methods have already achieved a comparatively high standard; however, there is still the objective to provide balloon catheters that not only are capable of being pushed forward over long distances but also follow a narrow and coiled configuration of blood vessels without difficulty.

As proposed by the present invention this objective is accomplished by providing a balloon catheter comprising an outer tube to which distal end a balloon connects that can be expanded by feeding in a fluid via the outer tube, wherein an inner tube extends at least partially through the outer tube, said inner tube passing through the balloon and terminating distally of the balloon, with the inner tube having a lumen appropriate to accommodate a guidewire and the balloon catheter having a distal and a proximal section, wherein the proximal section of the, outer tube is made of metal and the distal section consists of plastic material making the distal section more flexible than the proximal section, and wherein the distal section consists of a first distal section and a second distal section, said second distal section being located proximal to the first distal section, with the first distal section being made so as to be more flexible than the second distal section.

As proposed by the invention the distal section of the balloon catheter is thus divided into a first and a second distal section. The first distal section is arranged further distally than the second distal section, which means the second distal section abuts distally on the proximal section made of metal whereas the first distal section extends through those areas of the balloon catheter that are located farthest distally including the balloon itself. Since an additional graduation is made with respect to the softness, respectively flexibility of the distal section of the catheter the insertion capability is enhanced further, i.e. the first distal section makes sure that the balloon catheter portion that must navigate through the narrowest blood vessels is particularly soft. Nevertheless, for the entire length of the distal section such a soft material would be rather inappropriate because it would not be capable of being moved forward over longer distances. For that reason, a second distal section is provided and arranged between the first distal section and the proximal section, said second distal section ranging with respect to its softness/flexibility between that of the metallic proximal section and the especially soft first distal section. As used in the description the term softness refers to and means the flexibility of the relevant balloon catheter sections, i.e. their ability to adapt to the surrounding conditions. Therefore, the terms “soft” and “flexible” are equivalent in meaning.

The difference in softness of the first and second distal section can be achieved by using different plastic materials. Accordingly, the plastic material of the first distal section is softer than that of the second distal section.

Basically, various plastic materials respectively polymers may be employed for balloon catheters, for example polyethylene, polyurethane, polyvinyl chloride, polyamides, polyimides, silicones, polyether amides, polytetrafluoroethylene or EPDM rubber (ethylene propylene diene monomer).

Especially preferred and proposed by the invention for the first distal section is the use of thermoplastic elastomers, in particular polyether block amides (PEBA). This is a thermoplastic elastomer obtained by the polycondensation of a carboxylic acid polyamide with a polyether with terminal OH groups. In particular, PEBA is sold by the company of Arkema under the tradename of PEBAX®

Alternatively, other polyamides may also be used for the first distal section, in particular those available from the company of EMS-GRIVORY under the tradename of Grilamid®. Especially preferred is the use of a polyamide 12 (PA 12, Grilamid® L), polyamide obtained by polycondensation of laurolactam. Moreover, further conducively usable polyamides are polyamide 10.10 (PA 10.10, Grilamid® 1S), a polyamide obtained by polycondensation of decandiamine and sebacic acid, polyamide 6.10 (PA 6.10, Grilamid® 2S), polyamide obtained by polycondensation of hexamethylenediamine and sebacic acid, or polyamide 6.12 (PA 6.12, Grilamid® 2D), a polyamide obtained by polycondensation of hexamethylenediamine and dodecanedioic acid.

For the second distal section polyamides such as polyhexamethylene adipic acid amide (Nylon) can be used in particular. Although this material is also rather soft it is nevertheless less soft than the material employed for the first distal section, for example PEBA. Especially for the first distal section a material can be used that has a Shore D hardness in the range of between approx. 25 and 72. Appropriate and suitable for the second distal section are materials having a Shore D hardness ranging between 80 and 85.

Both with respect to the first and with respect to the second distal section the specific properties of the polymers may be adjusted by the addition of additives, softeners, filler substances, modifiers, and processing aids and agents. Basically, the respective substances are known to those skilled in the art.

To ensure the different materials have an altogether advantageous effect on the balloon catheter it is considered expedient to provide for the inner tube in the first distal section to be made of a material that is softer than that used in the second distal section. Typically, the inner and outer tube are each made of the same material. It is ensured in this way that in its first distal section the entire balloon catheter is clearly more flexible and can better adapt to the inner wall of the blood vessels than is the case in its second distal section.

As regards the first distal section it is to be noted that this section also includes the balloon itself which it typically made of another type of material than the inner and outer tubes in the area of the first distal section, for example of polyhexamethylene adipic acid amide (Nylon) which has proven its worth for the making of balloon catheters, or of another polyamide. The outer tube, however, that extends up to the balloon in distal direction and then merges into the balloon is made of a softer material in the first distal section, and the same is usually true for the inner tube in the first distal section, said tube passes through the balloon itself and extends further distally.

The term balloon as it is used within the scope of the present invention shall be understood to define the element of a balloon catheter that can be expanded by feeding in a fluid, irrespective of the form or material of said expandable element. With respect to its dimensions the catheter is suitably designed for the insertion into a body lumen, especially into a (blood) vessel system. The relevant dimensions of such catheters may vary depending on whether the blood vessel is, for example, a coronary artery, an intracranial blood vessel or an artery in the lower leg.

Normally, a guidewire extends through the inner tube, with said wire exiting the inner tube at the distal end of the balloon catheter. Various balloon catheter types are known basically, and a distinction is especially made between the over the wire technique (OTW catheter) and the rapid exchange technique (Rx catheter). Whereas in OTW catheters the guidewire extends through the entire volume, Rx catheters are provided with a passage opening allowing access to the inner tube via which the guidewire may exit proximally to the balloon but distally of the metallic hypotube. Advantageously, the balloon catheter proposed by the present invention is an Rx catheter, with the passage opening for the guidewire being situated in the second distal section. The outer tube in this place is provided with a passage opening serving as access to the inner tube, i.e. the inner tube terminates at this point which is also referred to as port.

An advantage of an Rx catheter is that due to the shorter guidewire lumen formed by the inner tube a replacement of the catheter via the guidewire is facilitated. In this manner, the proximal tip of the guidewire can be inserted into the distal opening of the guidewire lumen in the balloon catheter whereupon the catheter is pushed forward until the proximal tip of the guidewire again exits through the port of the balloon catheter. The attending physician may thus seize both the guidewire and the balloon catheter and in this way navigate the balloon catheter to the placement site along the guidewire. In an OTW catheter the guidewire would have to be of considerable length for this purpose which would also be associated with and give rise to sterilization problems. On the other hand, pushing an Rx catheter forward may under given circumstances be more difficult because due to the missing inner guidewire: there is no stiffening element in the proximal area of the catheter. However, this problem is solved with the present invention since it arranges for the proximal section to be provided as a metallic hypotube.

Another advantage offered by Rx catheters is that there is no need for a separate guidewire lumen in the proximal section of the balloon catheter. For that reason, the structure of the balloon catheter in this area is simpler and a lower cross sectional area can be provided. Moreover, the frictional forces arising between guidewire and guidewire lumen, i.e. the inner wall of the inner tube, are reduced because the guidewire for the most part extends parallelly to the balloon catheter and does not pass through the balloon catheter.

Whereas the inner tube constitutes the guidewire lumen the outer tube especially serves also as lumen through which fluid is supplied, i.e. the fluid, in particular a gas, is supplied for dilatation purposes to the balloon through the interior of the outer tube. This fluid supply lumen extends without interruption up to the balloon from the proximal section via the second distal section and the first distal section. The proximal section of the outer tube in particular is fabricated of stainless steel.

Obviously, the individual sections have to be connected with each other, especially the transitions between the first distal to the second distal section and from the second distal section to the proximal section can be made by welding. In other words, the different sections of the outer tube are welded together at their points of transition. The same applies to the inner tube, although the inner tube in an Rx catheter that is preferred by the invention terminates before the proximal section so that there is no need to create for the inner tube a transition between the second distal section and the proximal section. As alternatives to welding other connection methods may also be provided, for example adhesive methods. If thought expedient in view of establishing a secure connection the materials used for the different sections may slightly overlap at the transition points between the first and the second distal section as well as between the second distal section and the proximal section.

Typically, the balloon catheter proposed by the invention has a total length of more than 1 m, in particular more than 1.4 m. Preferred is a total length of approx. 145 cm which enables an access to be made in the groin region so that the balloon catheter can be navigated to many different placement sites including the intracranial area. The largest part of the total length is occupied by the proximal section which may even be longer than 1 m. By contrast, the length of the first distal section preferably amounts to 10 to 20 cm, in particular 14 to 15 cm, with the first distal section extending over the entire area from the distal tip of the balloon catheter to the transition to the second distal section. Typically, the latter has a length ranging between 10 and 30 cm, in particular between 15 and 25 cm. The distance between the passage opening in the second distal section for the inner tube and the proximal section typically amounts to approx. 20 to 40 mm, in particular is approx. 30 mm.

A typical outside diameter of the outer tube in the first distal section amounts 0.8 to 1.0 mm, in particular is approx. 0.9 mm. The inside diameter typically ranges between 0.7 and 0.8 mm. The outside diameter of the inner tube may for example amount to 0.5 to 0.6 mm, the inside diameter of the inner tube may range between 0.4 and 0.5 mm.

In the second distal section the outside diameter of the outer tube usually is slightly greater than that in the first distal section and typically ranges between 0.9 and 1.1 mm, in particular amounts to approx. 0.95 mm. The inside diameter of the outer tube in the second distal section in most cases ranges between 0.8 and 0.9 mm, whereas the dimensions of the inner tube largely coincide with those in the first distal section.

The balloon of the balloon catheter may be of drug-eluting type or may be uncoated. Drug-eluting balloons serve the purpose of preventing restenosis by having the balloon coated with a drug that is released and brought into contact with the inner wall of the vessel when the balloon is dilated. The active agent used is, in particular, a drug or medical substance that has a proliferation-inhibiting effect preventing a vasoconstrictive overgrowing of the vessel location previously expanded by the balloon. The active agent may in particular be selected from the following: Tretinoin, orphan receptor agonists, elafin derivatives, corticosteroids, steroid hormones, paclitaxel, rapamycin, tacrolimus, hydrophobic proteins as well as substances modifying cell proliferation. Mixtures of these active substances may also be used. Moreover, derivatives of the above cited active agents may also be employed, wherein said derivatives may in particular be salts, esters, and amides. As steroid hormones methylprednisolone, dexamethasone or estradiol may be used, for example. Especially preferred is the use of paclitaxel or paclitaxel derivatives. If thought expedient, a polysaccharide coating, particularly a dextran coating, may additionally be applied as has been described in publication WO 2012/072074 A1. In this way it is ensured that the active agent positively adheres to the inner vessel wall so that significant active agent concentrations that effectively prevent restenosis can sometimes be detected even months after the treatment has been administered.

Another expedient possibility is to apply an active agent to the balloon by repeatedly wetting the balloon with an active agent solution, with different solution agents being used sometimes. This results in the active agent coating as a whole to become more brittle causing the agent removal/abrasion to increase as has been disclosed in publication WO 2010/009904 A2. As per another variant of the active agent application the surface of the balloon is structured by mechanical, thermal or chemical methods resulting in the surface to be enlarged and provided with recesses the depth or width thereof typically ranging between 5 and 50 μm.

Over the length of the balloon catheter radiopaque markers may be arranged at various positions, said markers serving the purpose of making the catheter visible on radiographs. In particular, said markers may be manufactured of platinum or a platinum alloy.

At the proximal end of the balloon catheter a connection element is typically arranged that basically may have a conical form, for example a so-called Luer taper connection. At this point, a gas supplying device is hooked up that enables the balloon to be expanded. At its proximal end the guidewire extending wholly or partly through the balloon catheter is typically held by means of a torquer which facilitates handling the guidewire which is usually very thin.

In the framework of the description the term proximal shall be understood to be situated nearest to the attending physician, meaning the proximal end points into the direction external to the body. Vice versa, the distal end faces away from the physician, i.e. points towards the inside of the body.

Further elucidation of the invention is provided through the enclosed figure, where

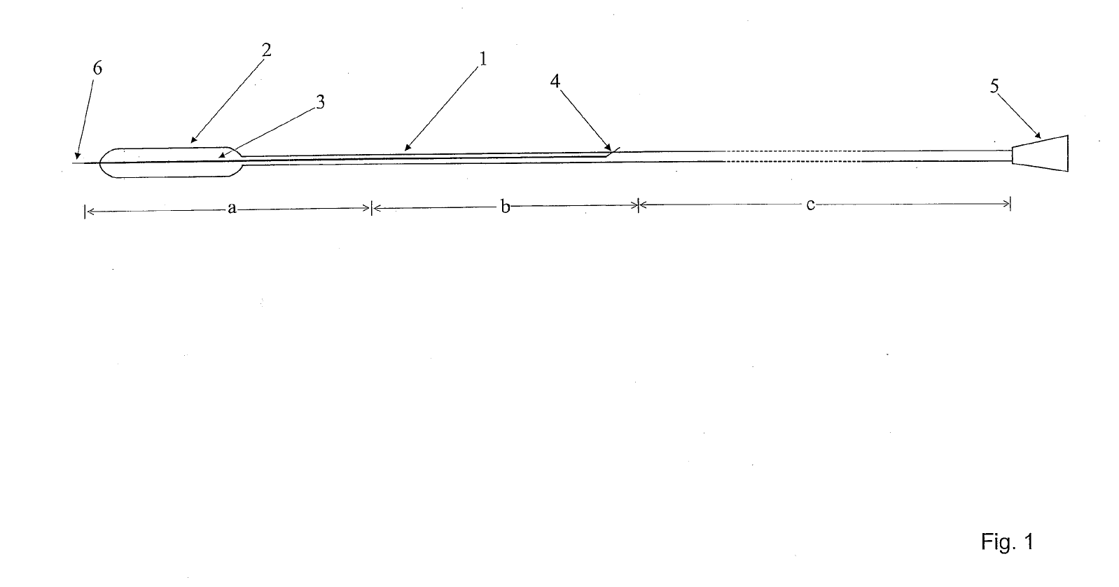

FIG. 1: is a side view of the balloon catheter according to the invention.

The inventive balloon catheter is provided with an outer tube 1 through which another inner tube 3 extends over given sections. At the proximal end of the balloon catheter a connecting element 5 is located whereas the distal end of the outer tube 1 transitions into balloon 2. With a view to expanding the balloon 2 the connecting element 5 is hooked up to a gas supplying device via which the balloon 2 will be dilated. Following this, the balloon 2 is again caused to collapse by withdrawing the fluid. The lumen via which the fluid is feed in extends through the outer tube 1.

The guidewire 6 can be run through the inner tube 3 and exits at the distal end of the balloon catheter. In the case of an Rx catheter the outer tube 1 is provided with a passage opening 4, also referred to as port, where the inner tube 3 terminates and through which the guidewire 6 proximally leaves the balloon catheter. From this point onwards, the guidewire 6 can further extend external to the balloon catheter in proximal direction and be seized by the attending physician outside the body with the help of a torquer.

Essentially, the balloon catheter comprises three sections: a first distal section a, a second distal section b, and a proximal section c. These are designed to differ with respect to their flexibility for which purpose different materials are used as a rule. The proximal section c is the longest and manufactured of a metal, in particular stainless steel. Abutting to this section is the second distal section b that is made of a soft but not extremely soft polymer material, in particular of nylon. Finally, the second distal section b extends further distally and transitions into the first distal section a; this section is designed so as to be especially flexible which is achieved by using for the fabrication of both the inner and the outer tubes 1,3 an especially soft material, in particular PEBA. The balloon 2 itself usually consists of nylon.

1. Balloon catheter comprising an outer tube (1) to which distal end a balloon (2) connects that can be expanded by feeding in a fluid via the outer tube (1), wherein an inner tube (3) extends at least partially through the outer tube (1), said inner tube (3) passing through the balloon (2) and terminating distally of the balloon (2), with the inner tube (3) having a lumen appropriate to accommodate a guidewire (6) and the balloon catheter having a distal (a,b) and a proximal section (c), wherein the proximal section (3) of the outer tube (1) is made of metal and the distal section (a,b) consists of plastic material making the distal section (a,b) more flexible than the proximal section (c), wherein the distal section (a,b) consists of a first distal section (a) and a second distal section (b), said second distal section (b) being located proximal to the first distal section (a), with the first distal section (a) being made so as to be more flexible than the second distal section (b).

2. Balloon catheter according to claim 1, wherein the outer tube (1) in the first distal section (a) is made of a softer plastic material than in the second distal section (b).

3. Balloon catheter according to claim 1, wherein the outer tube (1) in the first distal section (a) is made of a thermoplastic elastomer.

4. Balloon catheter according to claim 3, wherein the outer tube (1) in the first distal section (a) is made of a polyether block amide.

5. Balloon catheter according to claim 1, wherein the outer tube (1) in the first distal section (a) is made of a polyamide 12.

6. Balloon catheter according to claim 1, wherein the outer tube (1) in the second distal section (b) is made of a polyamide.

7. Balloon catheter according to claim 1, wherein the inner tube (3) in the first distal section (a) is designed so as to be more flexible than in the second distal section (b).

8. Balloon catheter according to claim 7, wherein the inner tube (3) in the first and in the second distal section (a,b) is in each case made of the same material as the outer tube (1).

9. Balloon catheter according to claim 1, wherein the outer tube (1) in the second distal section (b) is provided with a passage opening (4) allowing access to the inner tube (3).

10. Balloon catheter according to claim 1, wherein the proximal section (c) of the outer tube (1) is made of stainless steel.

11. Balloon catheter according to claim 1, characterized by a total length of more than 1 m.

12. Balloon catheter according to claim 1, characterized by a total length of more than 1.4 m.

13. Balloon catheter according to claim 1, wherein the length of the first distal section amounts to 10 to 20 cm.

14. Balloon catheter according to claim 1, wherein the length of the first distal section is 14 to 15 cm.

15. Balloon catheter according to claim 1, wherein the length of the second distal section amounts to 10 to 30 cm.

16. Balloon catheter according to claim 1, wherein the length of the second distal section is 15 to 25 cm.

17. Balloon catheter according to claim 1, wherein the outside diameter of the outer tube (1) in the first distal section (a) amounts to 0.8 to 1.0 mm.

18. Balloon catheter according to claim 1, wherein the outside diameter of the outer tube (1) in the second distal section (b) amounts to 0.9 to 1.1 mm.
